(19) United States
(12) Patent Application Publication
(10) Pub. No.: US 2026/0056195 A1
(43) Pub. Date: Feb. 26, 2026

(54) APPARATUS AND METHODS FOR ASSAYING A LIQUID SAMPLE

(71) Applicant: Smart Reader, LLC, Rockingham, VA (US)

(72) Inventors: William John HALL, Harrisonburg, VA (US); Andy STURMAN, San Diego, CA (US)

(21) Appl. No.: 19/189,647

(22) Filed: Apr. 25, 2025

Related U.S. Application Data
(63) Continuation of application No. PCT/US2023/036116, filed on Oct. 27, 2023.
(60) Provisional application No. 63/420,350, filed on Oct. 28, 2022.

(51) Int. Cl. G01N 33/543; G01N 21/47; G01N 33/53

(52) U.S. Cl. CPC G01N 33/54388; G01N 21/47; G01N 33/5302

(57) ABSTRACT

A reader may be configured to be lowered over a test strip cassette by hand by a user. A protruding portion of the bottom surface of the reader may engage a recessed portion of the top surface of the test strip cassette. A reader may comprise a reference sensor that receives light not reflected from the test strip.

### CROSS REFERENCE TO RELATED APPLICATIONS

This application is a continuation of PCT Application PCT/US2023/036116, filed on Oct. 27, 2023, which application claims priority to US Provisional Application 63/420,350 filed on Oct. 28, 2022. Both of these applications are hereby incorporated by reference in their entireties.

### BACKGROUND

It is often desirable in medicine or the biological sciences to be able to determine the presence or concentration of a particular target substance in a biological and/or liquid sample. While many methods of performing such assays are known, conventional methods often require the use of expensive equipment. This can meaningfully limit access to and cost effectiveness of certain scientific and/or professional practices that require, or are rendered more effective through, the use of such assays.

Devices and systems that can be used in the home by untrained consumers have been developed. These include, for example, commercially available pregnancy and ovulation test devices. In addition, it has become desirable to detect a wide variety of environmental conditions such as chemical contamination, mold, or the presence of other pests. Assays for these conditions may be performed by professionals or consumers, each with their own particular needs. Accordingly, systems and methods for performing assays at a reduced cost and/or with increased convenience are desirable.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In one implementation, a reader for a lateral flow assay comprises a housing, at least one light source associated with the housing, and at least one optical sensor associated with the housing. At least one sensing aperture in a lower portion of the housing is configured to allow light from the at least one light source to exit the lower portion of the housing and to allow light reflected from the lateral flow assay to enter the lower portion of the housing and illuminate the at least one optical sensor.

In another implementation, a lateral flow assay system comprises a lateral flow test strip cassette and a reader. The lateral flow test strip cassette comprises a housing and a lateral flow test strip associated with the housing. The reader comprises a housing, at least one light source associated with the housing, and at least one optical sensor associated with the housing. At least one sensing aperture in a lower portion of the housing is configured to allow light from the at least one light source to exit the lower portion of the housing and to allow light reflected from the lateral flow assay to enter the lower portion of the housing and illuminate the at least one optical sensor. An upper portion of the housing of the lateral flow test strip cassette comprises at least a first registration feature, and a lower portion of the housing of the reader comprises at least a second registration feature configured to engage with the at least a first registration feature in the upper portion of the housing of the lateral flow test strip cassette.

In another implementation, a method of assaying a liquid sample comprises hand-placing a test strip cassette on a substantially flat surface with a test line of a test strip inside the test strip cassette facing upward, hand-placing a reader onto the test strip cassette with a sensing aperture of the reader aligned with the test line of the test strip and obtaining an assay result from the reader.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1A is a block diagram of a lateral flow test strip in a cassette and an assay reader comprising light sources and detectors in a first orientation in accordance with some embodiments.

FIG. 1B is a block diagram of a lateral flow test strip in a cassette and an assay reader comprising light sources and detectors in a second orientation in accordance with some embodiments.

FIG. 1C is a flow chart of a first method of performing an assay of a liquid sample in accordance with some embodiments.

FIG. 1D is a flow chart of a second method of performing an assay of a liquid sample in accordance with some embodiments.

FIG. 2A is a top view of a lateral flow assay reader in accordance with some embodiments.

FIG. 2B is a bottom view of a lateral flow assay reader in accordance with some embodiments.

FIG. 3 is a side view of a lateral flow assay reader in accordance with some embodiments.

FIG. 4A is a perspective view of lateral flow assay cassette on a flat surface in accordance with some embodiments;

FIGS. 4B and 4C illustrate placing a reader over the lateral flow assay cassette of FIG. 4A.

FIG. 5A is a perspective view of another embodiment of a lateral flow assay cassette on a flat surface in accordance with some embodiments;

FIGS. 5B and 5C illustrate placing a reader over the lateral flow assay cassette of FIG. 4A in two different orientations.

FIG. 6A is a cross section showing engagement of registration features of a reader and a lateral flow assay cassette in accordance with some embodiments.

FIG. 6B is an exploded view of a reader in accordance with some embodiments.

### FIGS. 7A and 7B show perspective views of a case for a reader in accordance with some embodiments;

FIG. 8 illustrates another embodiment of a lateral flow assay system in accordance with some embodiments.

FIG. 9 illustrates another embodiment of a lateral flow assay system in accordance with some embodiments.

FIGS. 10A and 10B illustrate the lateral flow assay system of FIG. 9 with the reader and the test strip in a first orientation.

FIGS. 10C and 10D illustrate the lateral flow assay system of FIG. 9 with the reader and the test strip in a second orientation.

FIG. 11 illustrates another embodiment of a lateral flow assay system which can read a test strip in a plurality of different relative orientations.

FIG. 12 illustrates another embodiment of a lateral flow assay system in accordance with some embodiments.

FIGS. 13A and 13B illustrate a light shield that may be used with the lateral flow assay system of FIG. 12.

FIG. 14 illustrates an example component layout that may be used with the lateral flow assay system of FIG. 12.

FIG. 15 is an exemplary circuit schematic for the lateral flow assay system of FIG. 12.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

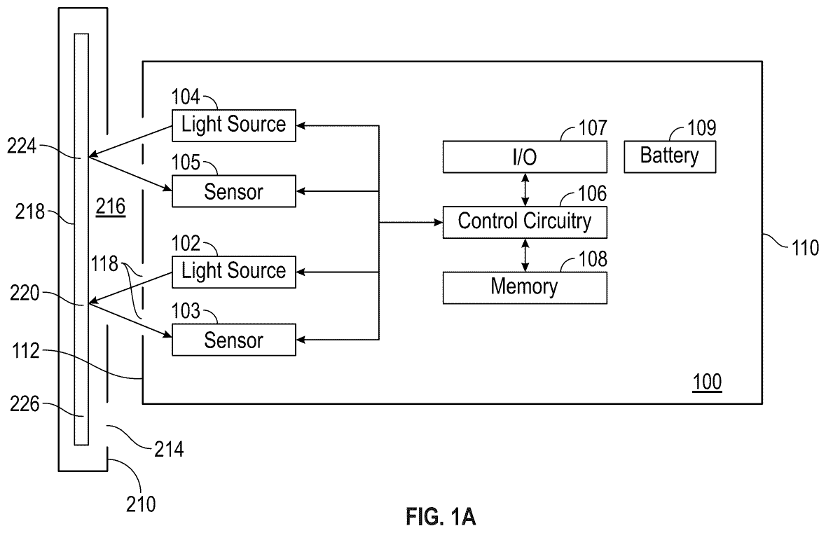

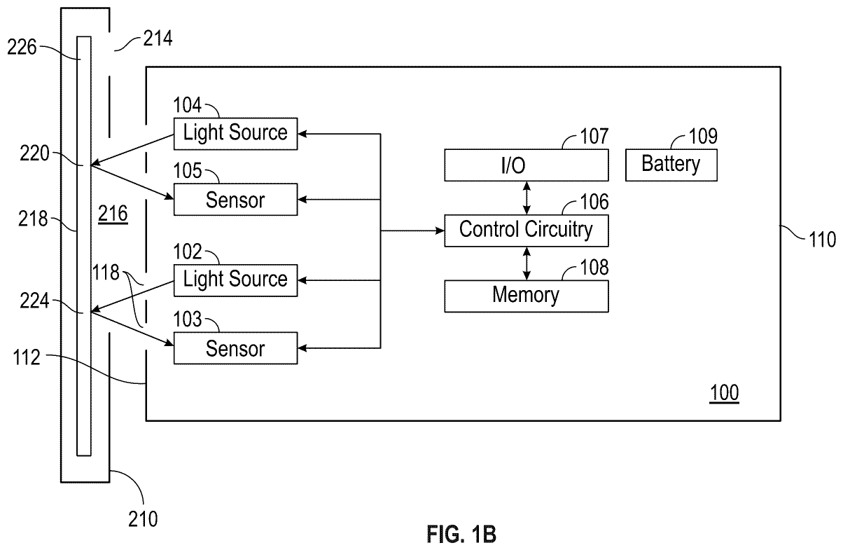

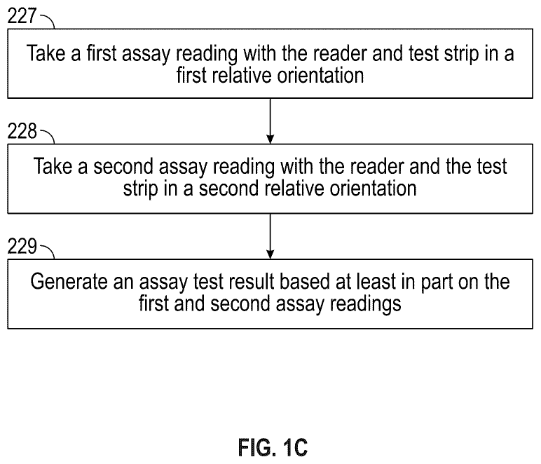

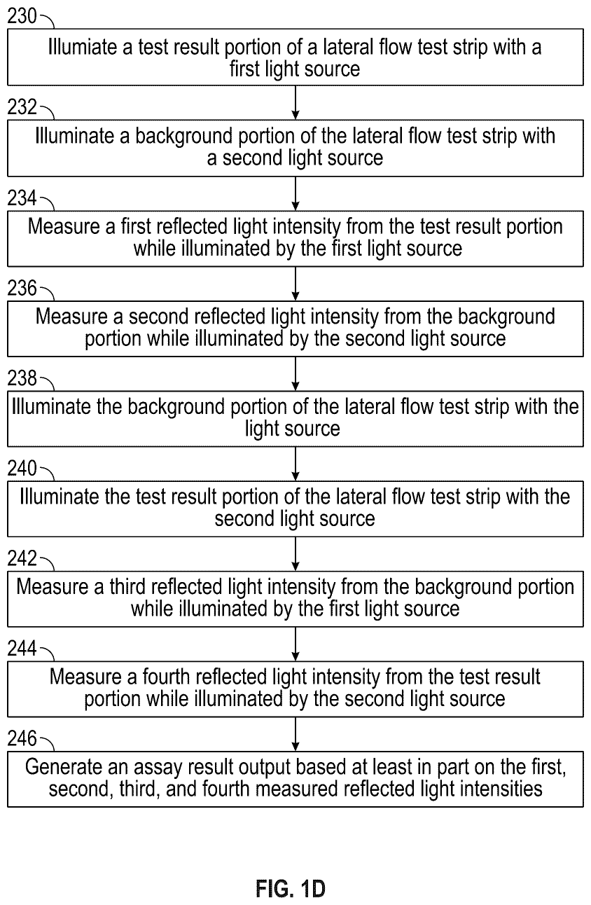

FIGS. 1A and 1B illustrate liquid sample assay apparatus in accordance with some embodiments. The apparatus comprises a lateral flow test strip reader 100 and a lateral flow test strip 218. The lateral flow assay test strip 218 comprises a test result portion 220 and a background portion 224. In the embodiment of FIG. 1A, the test strip 218 is provided as a test strip cassette with a housing 210 that at least partially encloses the test strip 218 and holds it in place within the cassette. The housing may comprise a first opening 214 that exposes a sample receiving portion 226 of the test strip 218 for sample application to the test strip 218. A user of the assay apparatus my provide a sample to be tested through the opening 214. Another opening 216 in the housing 210 of the cassette may be provided to expose the test result portion 220 and the background portion 224 of the test strip 218 for illumination and reading by the reader 100 as described further below.

The reader 100 comprises a housing 110 associated with a plurality of light sources, in this example two light sources designated 102 and 104 in FIG. 1A. The reader also comprises a plurality of sensors, in this example two sensors designated 103 and 105 in FIG. 1A. The sensors 103, 105 may, for example, be photodetectors. The light sources and sensors are coupled to and driven by control circuitry 106 which controls the operation of the light sources 102, 104 and receives output signals from the sensors 103, 105. The control circuitry may comprise an integrated circuit which may comprise a general-purpose microprocessor, digital signal processor, microcontroller, or the like. The control circuitry 106 may be connected to memory circuitry 108 that may be integral to a processor integrated circuit or separate therefrom. Input/output circuitry 107 may also be provided to deliver test results or other messages to users and may comprise LEDs, LCD displays, wireless communication circuits such as Bluetooth and the like. A battery 109 or other power source provides power for the electronic components of the reader 100.

In operation, light from a first light source 102 illuminates a portion of the test strip 218 through an aperture 118 in the housing 110 of the reader 100 which in the configuration of FIG. 1A is the test result portion 220 of the test strip. The aperture 118 may be in a cassette facing surface 112 of the housing 110. In some embodiments described further below, the cassette facing surface 112 may be considered a bottom surface of the housing 110, although this is not necessarily the case. The test result portion of the test strip is a portion which changes reflectivity in the presence of the substance to be detected by the assay. This reflectivity change may, as is well known, be generated by locally immobilized antibodies that bind or trap visible particles such as gold, latex, or the like when the substance of interest is in the sample applied to the test strip 218 on the sample receiving portion 226. The magnitude of the change in reflectivity may be dependent on the concentration of the substance of interest in the sample. Light from the light source 102 is reflected from the test result portion 220 of the test strip 218 and is collected through the same or a different aperture 118 in the cassette facing surface 112 of the reader housing 110 by a sensor 103. Typically, but not necessarily, the greater the concentration of the substance of interest in the sample the lower the reflectivity of the test result portion 220 of the test strip 218 and the lower the output signal of the sensor 103.

In addition, light from a second light source 104 illuminates a portion of the test strip 218 through an opening in the housing 110 of the reader 100 which in the configuration of FIG. 1A is the background portion 224 of the test strip. Light from the light source 104 is reflected from the background portion 224 of the test strip 218 and is collected through the same or a different opening in the cassette facing surface 112 of the reader housing 110 by a sensor 105. The background portion 224 may contain no immobilized antibodies such that visible particles are not bound or trapped in this area 224 of the test strip 218 during the test. For the background portion 224, there will be no change or essentially no change in reflectivity regardless of whether the substance of interest is present in the sample or not.

Although a wide variety of specific algorithms may be used, fundamentally, in the absence of the substance the difference between the outputs of sensors 103 and 105 are the same or similar, and in the presence of the substance the difference between the outputs of sensors 103 and 105 is relatively large. It will be appreciated however that a variety of sources of error exist in such systems and these errors can interfere with the correct interpretation or quantification of the results into an output indicating the presence, absence, or numerical concentration of the substance of interest in the sample.

The inventors have found that errors in these types of assay systems can be mitigated by performing the testing multiple times with different relative orientations of the test strip and the test strip reader. Furthermore, the inventors have found cassette and reader designs that make this error reduction process simple for a user of the system.

FIG. 1B illustrates the test strip 218 and reader 100 in a reversed orientation from that shown in FIG. 1A. In FIG. 1B, the sample application opening 214 is at the top, whereas in FIG. 1A the sample application opening 214 is at the bottom. The functional result of this reorientation is that the positions of the test result portion 220 of the test strip 218 and the background portion 224 of the test strip 218 are reversed. In FIG. 1B, the light source 102 and corresponding sensor 103 are measuring the reflectivity of the background portion 224 of the test strip 218 and the light source 104 and corresponding sensor 105 are measuring the reflectivity of the test result portion 220 of the test strip 218.

After repeating the test in both the FIG. 1A and FIG. 1B configurations, the two differences, e.g. background portion signal minus test portion signal in the first configuration and background portion signal minus test portion signal in the second configuration can be added or averaged to generate the final result. The inventors have found that this procedure not only reduces random error inherent in any measurement process but also reduces systematic errors that are common in these types of systems. For example, it is desirable to minimize component cost for the reader. Less expensive light sources and sensors generally have higher variability in output intensities and sensitivities. Differences in output intensity for light source 102 and light source 104 can introduce a systematic error in the measurement that is compensated for if the two measurements in different orientations are combined. Another example is that although shields are generally provided to keep ambient light from reflecting off the different areas of the test strip and entering the sensors, there is always usually some amount of ambient light leakage onto the test strip. The amount of ambient light reflecting off the test strip may be different on different areas of the test strip, and this can also introduce a systematic error that may be compensated for by performing the test a plurality of times in a plurality of different orientations and combining the results.

FIG. 1C is a flowchart illustrating an embodiment of a method performed in accordance with theses principles. At block 227, a first assay reading is taken with the reader and the test strip in a first relative orientation. At block 228 a second assay reading is taken with the reader and the test strip in a second relative orientation. At block 229, an assay test result is generated based at least in part on both the first and second assay readings.

FIG. 1D is a block diagram of the method of FIG. 1C in an embodiment where the reader has two light sources and two light sensors such as shown in FIGS. 1A and 1B. In this embodiment, at block 230 a test result portion of a lateral flow test strip is illuminated with a first light source. At block 232, a background portion of the lateral flow test strip is illuminated with a second light source. At block 234 a first reflected light intensity from the test result portion is measured while the test result portion is illuminated by the first light source and at block 236 a second reflected light intensity from the background portion is measured while the background portion is illuminated by the second light source. As one example embodiment, blocks 230, 232, 234, and 236 are being implemented by a reader and test strip in the orientation of FIG. 1A.

Moving to block 238, the background portion of a lateral flow test strip is illuminated with the first light source. At block 240, the test result portion of the lateral flow test strip is illuminated with the second light source. At block 242 a third reflected light intensity from the background portion is measured while the background portion is illuminated by the first light source and at block 244 a fourth reflected light intensity from the test result portion is measured while the test result portion is illuminated by the second light source. As one example embodiment, blocks 238, 240, 242, and 244 are being implemented the reader and test strip in the orientation of FIG. 1B.

The improved accuracy benefits of the invention as described above can be obtained with a single light source and associated detector as well. For example, rather than reversing the relative orientations of the reader and test strip as shown in FIGS. 1A and 1B, the relative orientation can be shifted or otherwise adjusted such that the same light source and sensor first measure the test result portion and then measure the background portion (or vice versa).

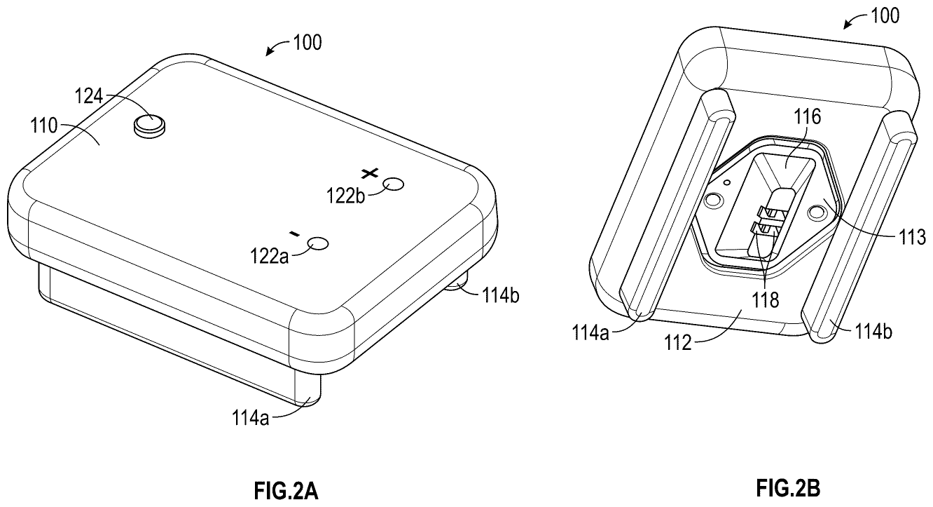

FIGS. 2A and 2B illustrate an embodiment of a reader 100 that has a variety of advantages over conventional test strip readers, including, among others, the ability to easily implement the orientation changes illustrated and described above with reference to FIGS. 1A through 1D. Referring now to FIG. 2A, the reader 100 comprises a housing 110 with standoffs 114a and 114b that in normal use would support the housing 110 off a support surface such as a tabletop, desk, or the like. The embodiment of FIGS. 2A and 2B shows two elongated standoffs but any number or configuration of one or more standoffs could be suitable. The surface opposite the standoffs 114a, 11b may be considered the top or upper surface of the housing 110 and may comprise a switch or any other user actuatable mechanism 124 to initiate an assay test procedure. The upper surface may also comprise visible output indicators for positive and negative test result indicators or numerical outputs indicative of a concentration of a substance of interest. These may be LEDs or ports through which LEDs can be seen 122a, 122b, an LCD display, or any other result output hardware. In some embodiments described further below, the test result output hardware may comprise in addition or as an alternative to the visual displays described above wireless communication circuitry such as Bluetooth, WiFi, or other transmission protocol.

FIG. 2B illustrates the lower or bottom surface of the housing, also illustrating the standoffs 114a, 114b. As will be described further below, the lower surface of FIG. 2B may comprise a test strip facing surface 112. One or more apertures 118 are provided in the test strip facing surface 112 for light from one or more light sources associated with the housing 110 to illuminate portions of the test strip and for reflected light to be collected by one or more optical sensors associated with the housing 110. In some advantageous embodiments, the apertures 118 are provided in a protruding portion 116 of the test strip facing surface 112. In some embodiments such as the one shown in FIG. 2B, the test strip facing surface comprises two parts, one part integral with the remainder of the housing 110 and a second part 113 that includes the protruding portion 116. It will be appreciated that as an alternative the protruding portion can be made an integral part of the remainder of the housing 110. In the embodiment of FIG. 2B, the protruding portion 116 has one or more sloped sides. In some embodiments, in some embodiments, the protruding portion 116 may be configured as a truncated pyramid.

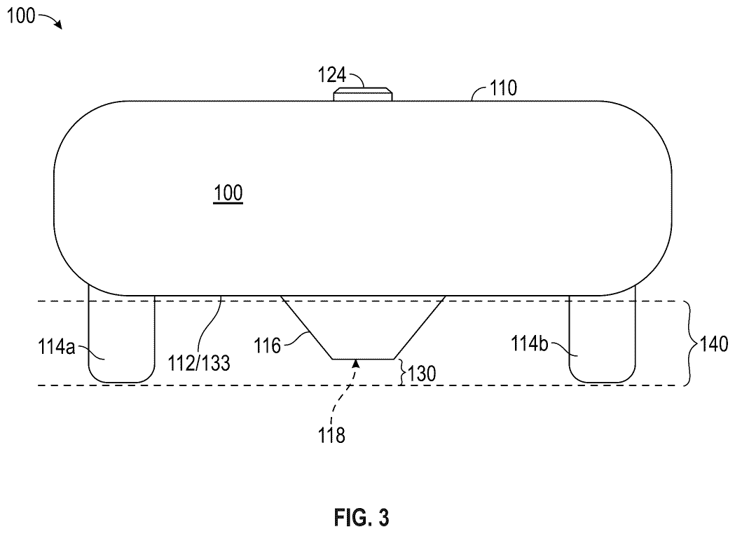

FIG. 3 shows a side view of the reader of FIGS. 2A and 2B. As can be seen in this figure, the height 140 of the standoffs 114a, 114b is greater than the downward extent of the protruding portion 116 such that when the reader 100 rests on a flat surface on the standoffs 114a, 114b a gap 130 is present between the bottom of the protruding portion 116 of the test strip facing surface 112 and the surface on which the standoffs 114a, 114b rest.

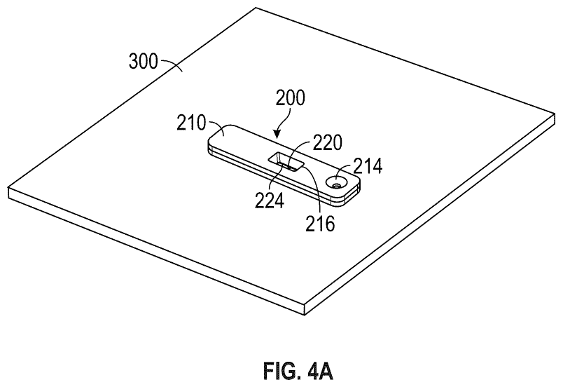

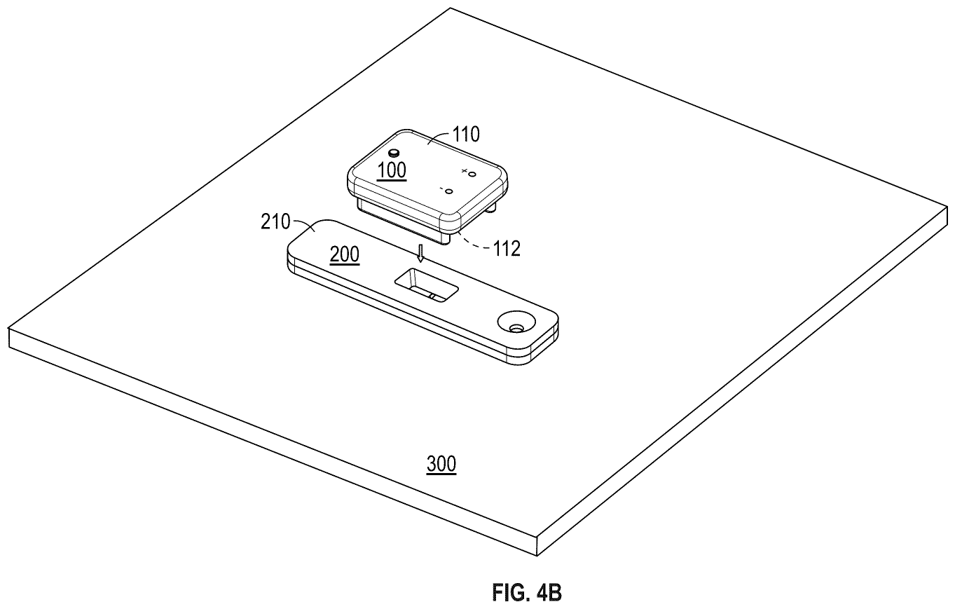

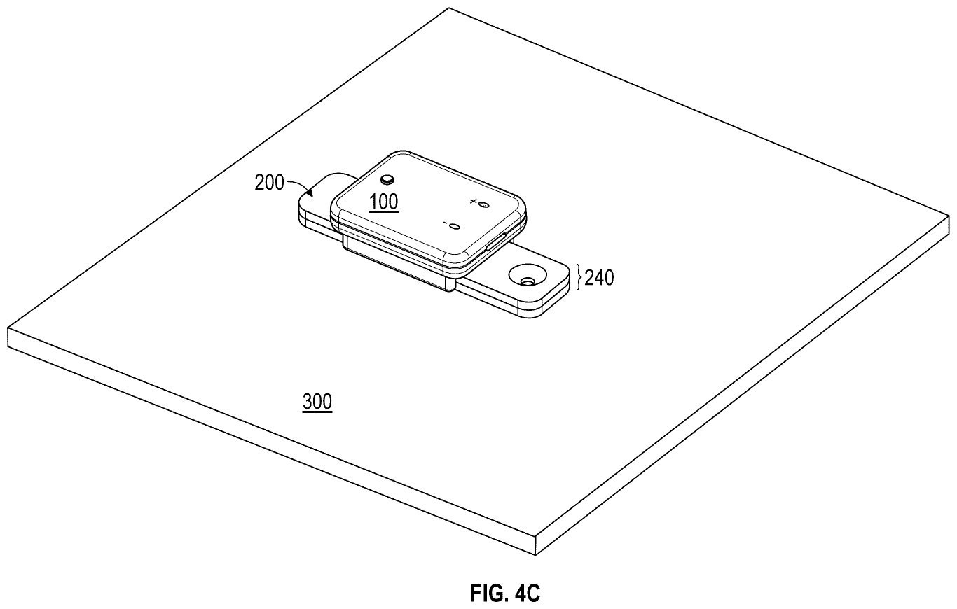

FIGS. 4A through 4C illustrate use of the reader 100 of FIGS. 2A, 2B and 3 with a test strip cassette 200. Referring first to FIG. 4A, a test strip cassette 200 comprising a housing 210 rests on a flat surface 300 of, for example, a table top, a desk, a lab bench, or the like. As is similarly shown in FIGS. 1A and 1B, a test strip is contained inside the housing 210 and comprises a test result portion 220 and a background portion 224. An opening 216 in the housing 210 of the cassette is provided to expose the test result portion 220 and the background portion 224 of the test strip 218 for illumination and reading by the reader 100 as described further below. The opening 216 may have inwardly sloping sides from the opening toward the test strip that match the sloping sides of the protruding portion 216 of the reader. A sample application port 214 is also provided.

FIGS. 4B and 4C illustrate how the reader 100 of FIGS. 2A, 2B, and 3 can be placed by a user onto the test strip cassette 200 with the test strip facing surface 212 of the reader housing 110 adjacent to the top surface of the cassette. The protruding portion 116 of the reader 100 may rest in the opening 216, with the sloping sides of the protruding portion in mating engagement with the sloping sides of the opening 216. As can be seen in FIGS. 4A and 4B, the test result portion 220 is a darkened line, indicating the presence of the substance of interest, so in FIG. 4C the LED output for a positive result is activated.

Referring again to FIG. 4C, the test strip cassette housing 210 has a height 240. This height 240 is advantageously greater than the height 140 of the standoffs (see FIG. 3) such that when the reader 100 is engaged with the test strip cassette 200 the housing 110 of the reader 100 is supported by the housing 210 of the test strip cassette 200 rather than being supported by the standoffs 114, particularly by engagement of the sloping walls of the protruding portion 116 of the reader 100 with the sloping walls of the opening 216 in the test strip cassette 200. This helps maintain a known and consistent distance between the surface of the test strip 218 and the light sources and sensors associated with the housing of the reader 100. In this embodiment, the main function of the standoffs 114 is to protect the protruding portion 116 from damage or foreign substances during handling and use. For example, with the standoffs 114, a user can set the reader 100 onto a surface without a cassette 200 underneath without having the protruding portion 116 with the sensing apertures 118 come into contact with the surface the reader is resting on. The standoffs 114 therefore may be beneficial but are not necessary components of the assay system.

The assay reader of FIGS. 2A, 2B, and 3 has several advantages over conventional lateral flow test strip readers. It can be made small, inexpensive, and reusable. It does not require the insertion of a test strip so it is very easy to perform a series of assays with a series of test strips. For example, a user could run a plurality of assays such as 2, 5, 10, or more assays with different samples and lay the corresponding plurality of test strips on a table, desk, lab bench or the like. To read the results, the user may simply place a reader 100 on top of each test strip and read each result. This minimizes test strip handling, increasing efficiency and reducing the chance of user error from improper insertion of a test strip cassette into an opening in a conventional reader.

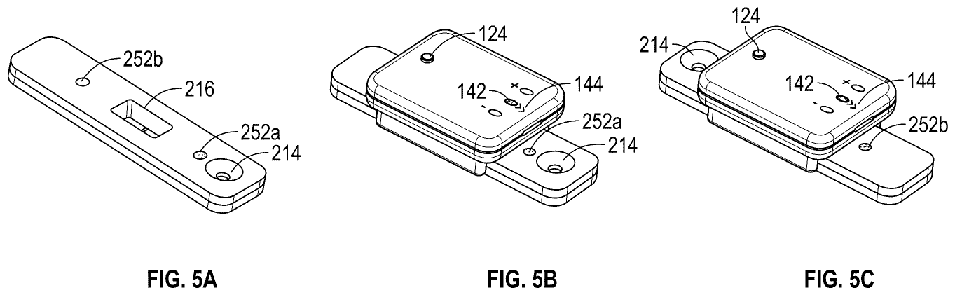

FIGS. 5A, 5B, and 5C illustrate how embodiments of the reader 100 of FIGS. 2A, 2B, and 3 can be easily used to take advantage of the reorientation methods for accuracy improvement described above. Fundamentally, the procedure is simply to place the reader 100 over the test strip cassette 200 in a first orientation such as shown in FIGS. 4B and 4C. Then, rotate either the reader 100 or the test strip cassette 200 by 180 degrees and place the reader 100 on the test strip cassette a second time. The second time, the light sources and sensors have swapped which part of the test strip they are adjacent to.

The embodiment shown in FIGS. 5A, 5B, and 5C includes features to direct a user when performing such a test protocol with a plurality of orientations between the reader and the test strip. FIG. 5A illustrates a test strip cassette which in addition to a sample application port 214 and a result viewing window 216 includes two markings 252a and 252b that differentiate the two ends of the test strip cassette. In FIG. 5A, these are illustrated as dots of different color such as red and green but any type of differentiating feature may be used including numbers, letters, embossed features on the cassette housing 210, etc.

As shown in FIG. 5B, the reader in this embodiment also includes an output 142 which indicates to the user the relative orientation that the user should place the reader on the cassette in. In the embodiment of FIG. 5B the output 142 is a green LED, which may be activated when the user turns the reader on with switch 124. The green LED activation indicates to the user that the user should place the green LED adjacent to the green marking 252a on the test strip cassette. After the user places the reader on the cassette, the reader takes a measurement in this orientation. After that measurement process is complete, the control circuitry in the reader may change the output 142 to red by activating a red LED also visible via the output 142. In response to this change, the user may reverse the relative orientation of the reader and the test strip cassette as shown in FIG. 5C. In the second relative orientation shown in FIG. 5C, the red output 142 is adjacent to the red marker 252b on the test strip cassette. This reverses the locations of the light sources and sensors over the test strip regions as described above. Another measurement is taken in this configuration. The two measurements are combined with a suitable algorithm and the result is output from the +or − outputs such as shown in FIG. 4C.

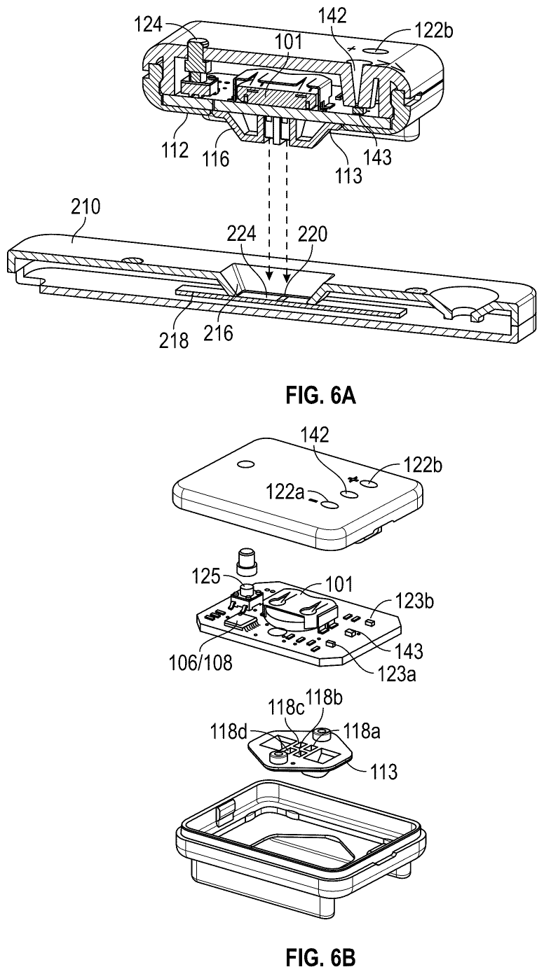

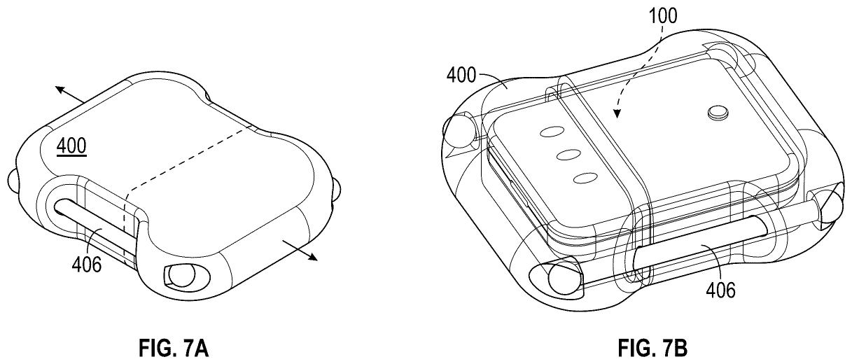

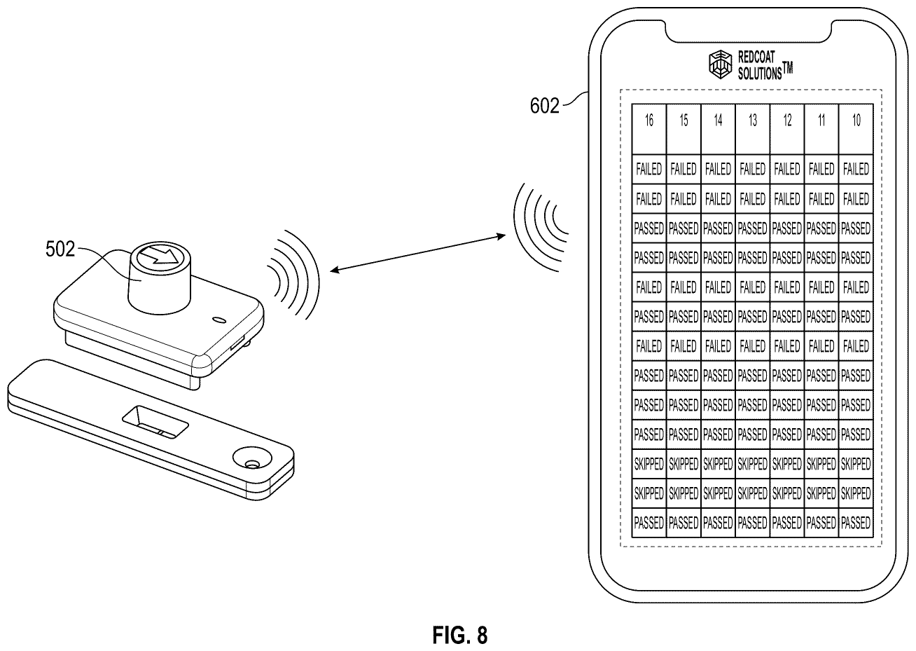

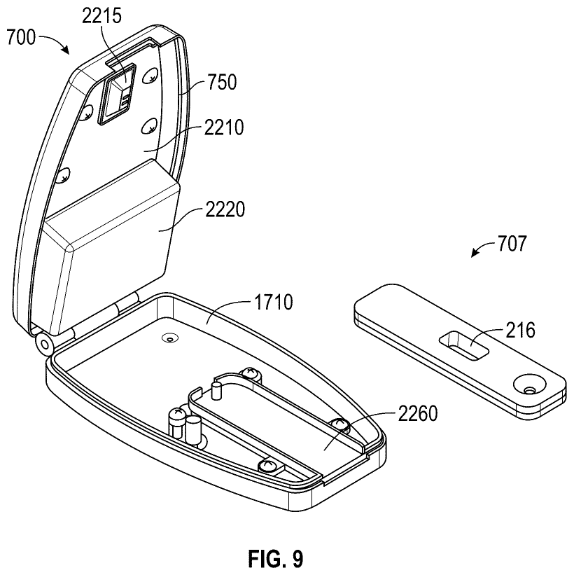

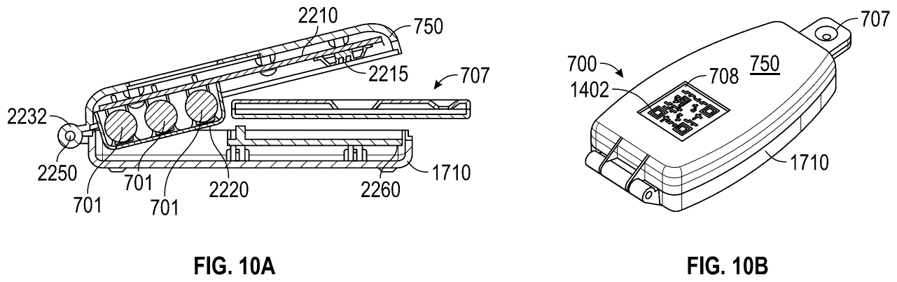

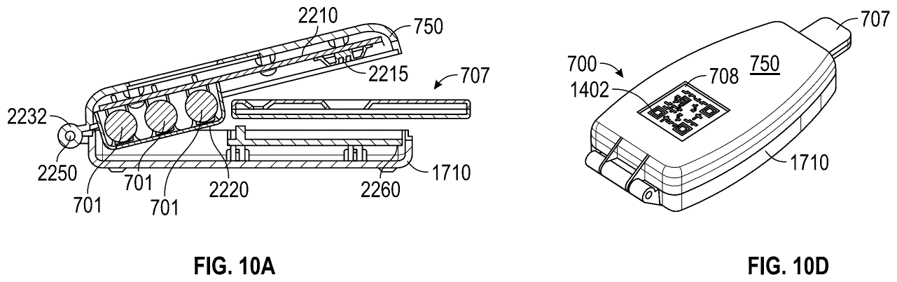

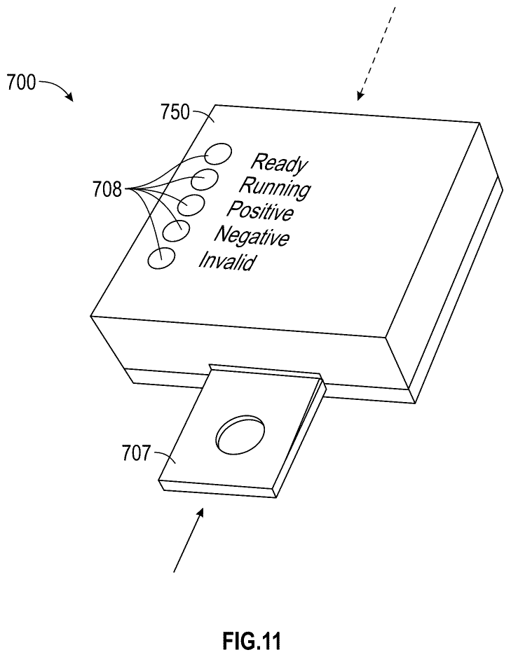

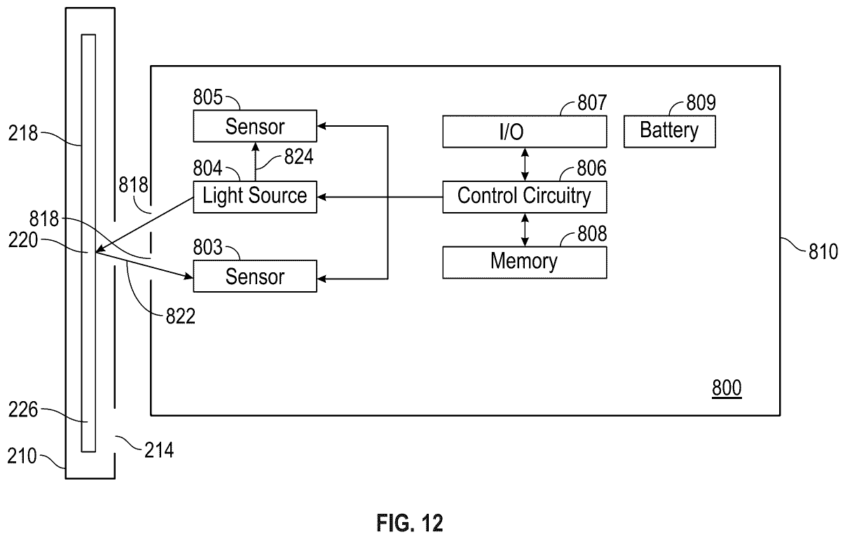

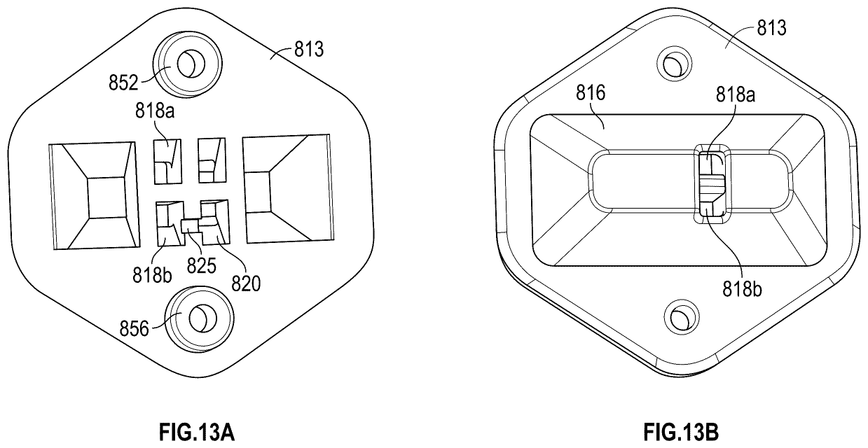

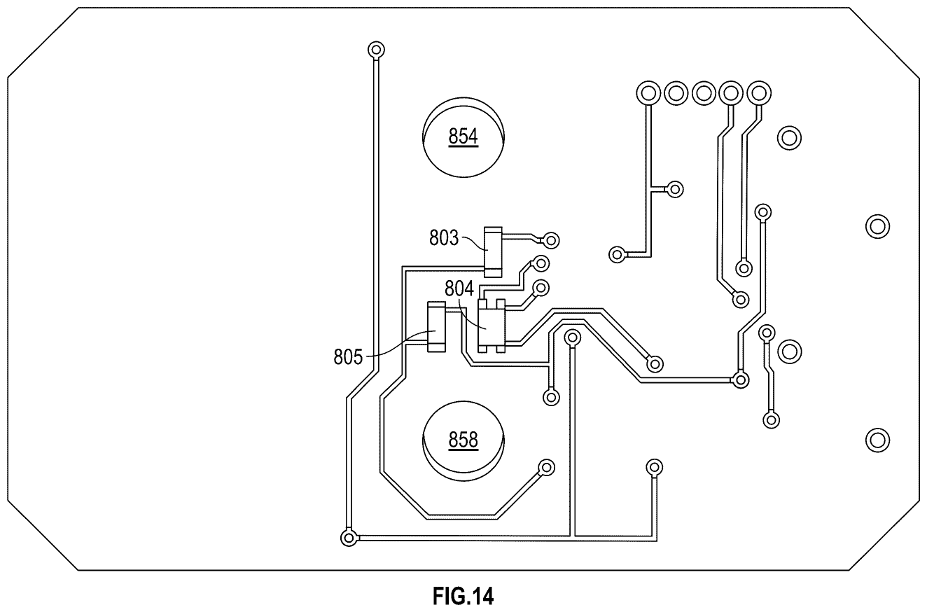

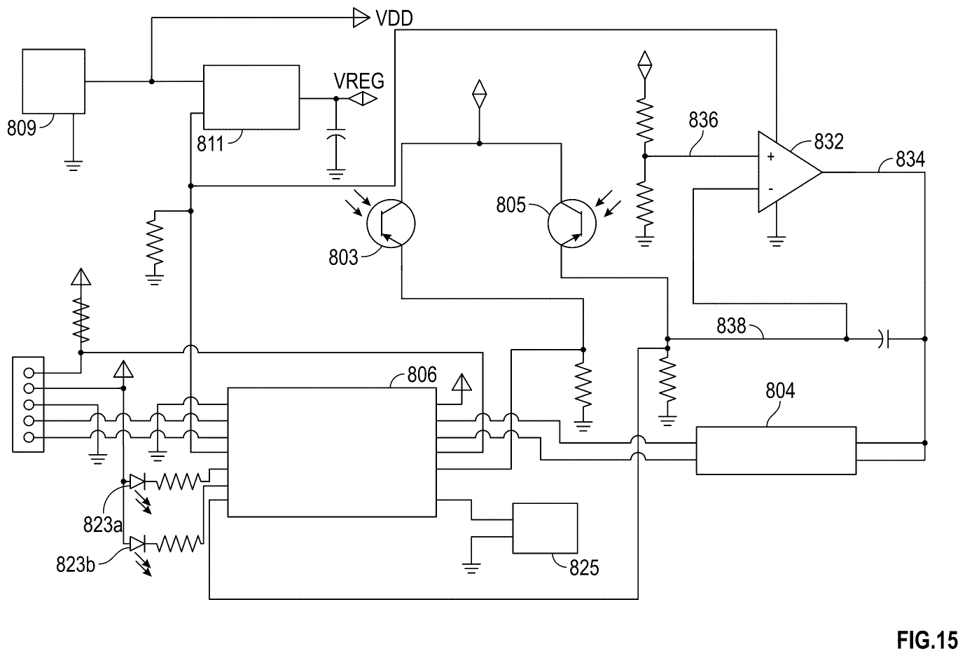

FIG. 6A shows a cross section of the reader and test strip cassette of FIGS. 5A, 5B, and 5C as the reader is lowered over the test strip cassette in the orientation illustrated in FIG. 5B. FIG. 6B is an exploded view of the reader of FIGS. 5B and 5C. As can be seen in FIGS. 6A and 6B, a printed circuit board inside the reader housing has mounted thereon a battery 101, control circuitry and memory 106, 108. Test result LEDs 123a, 123b are visible through openings 122a, 122b in the housing. The LED that functions as an orientation indicator 143 is visible through opening 142 in the housing. Button 124 is coupled to a miocroswitch 125 also mounted to the printed circuit board.

A light shield 113 is attached to the bottom of the printed circuit board with openings 188a, 118b, 188c, and 118d positioned over a first light source, a first sensor, a second light source, and a second sensor respectively. The openings 118a, 118b, 118c, and 118d communicate with openings in the lower surface of the protruding portion 116 of the light shield. When the reader is placed over the test strip cassette in the orientation illustrated in FIG. 6A, openings 118a and 118b are aligned with the test result region 220 of the test strip 218 and openings 118c and 118d are aligned with the background portion 224 of the test strip 218. When the reader is placed of the test strip cassette in the reversed orientation of FIG. 5C, these alignments are reversed.

FIGS. 7A and 7B illustrate a package for the reader of any of the above figures. The package 400 comprises two mating parts coupled longitudinally with elastic connectors. The two parts can be pulled apart by stretching the connectors longitudinally, releasing the reader 100 from the package.

FIG. 8 illustrates an embodiment wherein the reader is provided with wireless communication circuitry such as Bluetooth for communicating test results to an external device such as a smartphone. Two-way communication could be provided. In the embodiment of FIG. 8, a handle 502 is provided to facilitate user manipulation of the reader when placing the reader on the test strip cassette. The handle 502 may comprise an orientation indicator such as a small display (e.g. that can display an arrow pointing one direction or the other) or LEDs such as described above. For embodiments intended to be used in a single orientation only, the orientation indicator may be a fixed marker, embossed region, printed arrow, etc.

FIG. 9 illustrates another embodiment of a reusable reader 700 that can be used in different relative orientations with respect to the test strip to provide the error reduction/accuracy improvements described above. In this embodiment, the reader comprises a hinged top portion or lid 750 that can open with respect to a base shell 1710. Base shell 1710 comprises a test strip cassette mounting structure 2260 configured to receive cassette 707 such that at least an end portion of test strip cassette 707 extends outside of apparatus 700. A printed circuit board (PCB) 2210 is disposed on an underside of lid 750. Battery cover 2220 is configured to be disposed over a top (or bottom) and sides of batteries 701. As with the reader embodiments described above, a protruding light shield 2215 extends from the bottom of the printed circuit board 2210 having a shape configured to mate with the opening 216 in the test strip cassette housing. When the lid 750 is lowered over the base 1710, the protruding light shield 2215 becomes seated in the opening 216 of the test strip cassette 707 similar to the embodiments described above.

As seen in the cutaway view of FIG. 10A, apparatus 700 comprises base housing portion 1710 having a hinge 2232 with hinge pin 2250. As shown in FIG. 10B, this reader embodiment comprises an LCD display 708 for outputting test results and/or other information to the user, which may in some embodiments be in the form of a QR code 1402.

FIGS. 10A and 10B illustrate the test strip cassette 707 in a first orientation with respect to the reader 700. In this orientation, the sample application opening is outside the reader after insertion of the test strip cassette 707. In contrast, FIGS. 10C and 10D illustrate a reversed relative orientation of the test strip cassette 707 and reader 700. In the orientation of FIGS. 10C and 10D, the test strip cassette is inserted in a reversed orientation such that the sample application port is inside the reader. In embodiments such as shown in FIGS. 9 and 10A through 10D, the user may first insert the test strip cassette with a first end positioned internally to the reader. The reader may then make a measurement in that orientation. The user may then remove the test strip cassette form the reader and re-insert is with the second end positioned internally to the reader. The reader may then take a second measurement in the second orientation. The reader may then combine the two measurements to generate a result output to the user as described above.

Another method of taking measurements in different relative orientations of reader and test strip cassette is illustrated in FIG. 11. In this embodiment, the test strip cassette 707 is inserted into a receiving slot in the reader housing 700 as indicated by the solid arrow. After an assay measurement is taken with the test strip inserted in this orientation, the test strip may be inserted into a slot on the other side of the reader as indicated by the dashed arrow, again with the sample port on the outside of the reader after the test strip cassette is inserted. The depth of insertion in each direction can be controlled with stops or other mechanical features so that in the second orientation the light sources and sensors are swapped with respect to which portions of the test strip they are reading as described above with respect to other embodiments.

Accordingly, there are a variety of ways re-orientation can be made to occur between a reader and a test strip to obtain the accuracy improvements described herein.

FIG. 12 illustrates another liquid sample assay apparatus in accordance with some embodiments. Similar to the embodiment described above with reference to FIG. 1A, the apparatus comprises a lateral flow test strip reader 800 and a lateral flow test strip 218. The lateral flow assay test strip 218 comprises a test result portion 220. The test strip 218 is provided as a test strip cassette with a housing 210 that at least partially encloses the test strip 218 and holds it in place within the cassette. The housing may comprise a first opening 214 that exposes a sample receiving portion 226 of the test strip 218 for sample application to the test strip 218. A user of the assay apparatus may provide a sample to be tested through the opening 214. Another opening in the housing 210 of the cassette may be provided to expose the test result portion 220 of the test strip 218 for illumination and reading by the reader 800 as described further below.

The reader 800 comprises a housing 810 associated with at least one light source 804. The reader also comprises a plurality of sensors, in this example two sensors designated 803 and 805 in FIG. 12. The sensors 803, 805 may, for example, be photodetectors. The light source(s) and sensors are coupled to and driven by control circuitry 806 which controls the operation of the light source(s) 804 and receives output signals from the sensors 803, 805. The control circuitry may comprise an integrated circuit which may comprise a general-purpose microprocessor, digital signal processor, microcontroller, or the like. The control circuitry 806 may be connected to memory circuitry 808 that may be integral to a processor integrated circuit or separate therefrom. Input/output circuitry 807 may also be provided to deliver test results or other messages to users and may comprise LEDs, LCD displays, wireless communication circuits such as Bluetooth and the like. A battery 809 or other power source provides power for the electronic components of the reader 100.

In operation, light from the light source 804 illuminates the test result portion 220 of the test strip 218 through an aperture 818 in the housing 810 of the reader 800. The aperture may be in a cassette facing surface of the housing 810. As described above, the test result portion 220 of the test strip is a portion which changes reflectivity in the presence of the substance to be detected by the assay. This reflectivity change may, as is well known, be generated by locally immobilized antibodies that bind or trap visible particles such as gold, latex, or the like when the substance of interest is in the sample applied to the test strip 218 on the sample receiving portion 226. The magnitude of the change in reflectivity may be dependent on the concentration of the substance of interest in the sample. Light 822 from the light source 102 is reflected from the test result portion 220 of the test strip 218 and is collected through the same or a different aperture 818 of the reader housing 810 by a sensor 803. Typically, but not necessarily, the greater the concentration of the substance of interest in the sample the lower the reflectivity of the test result portion 220 of the test strip 218 and the lower the output signal of the sensor 803.

In contrast with the embodiment of FIGS. 1A and 1B, the embodiment of FIG. 12 illuminates only the test result portion 220 of the test strip 218. Rather than having a separate light source that illuminates a background portion of the test strip 218 and collecting the light reflected from the background portion of the test strip 218 with the second sensor, in the embodiment of FIG. 12 the second sensor detects light 824 from the light source 804 that is not reflected from the test strip 218. The output from the sensor 805 that detects the light 824 that is not reflected from the test strip 218 may be used to control the illumination intensity output from the light source 804 to create accurate and repeatable measurements of the test result portion 220 of the test strip 218 without making a measurement of a background portion of the test strip 218 and consequently without making measurement in forward and backward orientation such as described above. This reduces the number of components and cost associated with manufacturing the reader without sacrificing accuracy.

The embodiment of FIG. 12 can, like the embodiment of FIGS. 1A and 1B, be packaged in a housing essentially as shown above in FIGS. 2A, 2B, 3, 6A, and 6B. It can also be used in an analogous but simplified single measurement manner.

FIGS. 13A and 13B show a light shield 813 comprising a protruding portion 816 that is analogous to the light shield 113 of FIGS. 6A and 6B above but that is adapted for use with the embodiment illustrated in FIG. 12. In this light shield 813, opening 818a is the light 822 collection opening for the sensor 803 that collects light reflected from the test strip 218. Opening 818b is the illumination opening for the light source 804. Also provided in this light shield 813 is a closed well 820 in which the second sensor 805 sits. A channel 825 connects the opening 818b and the well 820 to allow light 824 from light source 804 that is not reflected from the test strip 218 to reach and be detected by the second optical sensor 805. FIG. 14 shows an example printed circuit board with the light source 804 and light sensors 803 and 805 mounted on a surface thereof. In this example, other electronic components shown in FIG. 12 may be mounted on the other side of this printed circuit board. As an assembly, the light shield 813 of FIG. 13A is turned over as shown in FIG. 13B and boss 852 engages opening 854 in the printed circuit board and boss 856 engages opening 858 in the printed circuit board. This aligns the light source with opening 818b, the sensor 803 with opening 818a, and the second sensor 805 with the well 820.

FIG. 15 is a schematic showing one example configuration and functionality of the components illustrated in FIG. 12. The schematic of FIG. 15 includes the light source 804, first sensor 803, second sensor 805, controller 806, battery 809, and I/O 807 in the form of result output LEDs 823a and 823b and wake/power up switch 825. Also present is a voltage regulator 811.

In the embodiment of FIG. 15, a control loop comprising op-amp 832 is provided. The op-amp output at node 834 provided the positive voltage input driving the light source 804. In operation, light from the light source 804 hits sensor 805 causing current to flow through R2 and raising the voltage at node 838 which is provided as the negative input to the op-amp 832. The output voltage of the op-amp at node 834 will stabilize at the light source 804 driving voltage that makes the voltage at node 838 equal to the positive op-amp input derived from the R3/R 8 voltage divider. This control loop produces a precise and consistent illumination intensity from the light source 804 regardless of differences that may be present in different light source components due to manufacturing tolerances, aging, or any other reason.

In the embodiment of FIGS. 14 and 15, the light source 804 is a single package that can separately deliver red or green light so that measurements of the test result portion of the test strip 218 can be made at two different wavelengths. The control loop described above will function to make the light source output intensity equal for both wavelengths.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,”etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

1-10. (canceled)

11. A reader for a lateral flow assay, the reader comprising:
a housing;
at least one light source associated with the housing;
at least one optical sensor associated with the housing;
at least one sensing aperture in a lower portion of the housing configured to allow light from the at least one light source to exit the lower portion of the housing and to allow light reflected from the lateral flow assay to enter the lower portion of the housing and illuminate the at least one optical sensor.

12. The reader of claim 11, wherein the at least one sensing aperture is located in a protruding portion of the lower portion of the housing.

13. The reader of claim 12, wherein the lower portion of the housing comprises a first lower portion and a second lower portion, and wherein the protruding portion is part of the second lower portion.

14. The reader of claim 13, wherein the second lower portion is integral with the first lower portion.

15. The reader of claim 13, wherein the second lower portion is a separate piece from the first lower portion.

16. The reader of claim 11, wherein the at least one light source and the at least one optical sensor are enclosed within the housing.

17. The reader of claim 11, wherein the at least one sensing aperture comprises at least two sensing apertures.

18. The reader of claim 11, wherein the at least one sensing aperture comprises at least four sensing apertures.

19. The reader of claim 11, wherein the at least one sensing aperture is formed in a light shield.

20. The reader of claim 19, wherein the light shield forms a protruding portion of the lower portion of the housing.

21. (canceled)

22. The reader of claim 12, wherein the protruding portion is shaped as a truncated pyramid.

23. The reader of claim 11, wherein the reader comprises feet that protrude from the lower portion of the housing.

24. The reader of claim 23, wherein the at least one sensing aperture is located in a protruding portion of the lower portion of the housing, and wherein the feet protrude farther from the lower portion of the housing than the protruding portion in which the at least one sensing aperture is located.

25-29. (canceled)

30. A method of assaying a liquid sample, the method comprising:
hand-placing a test strip cassette on a substantially flat surface with a test line of a test strip inside the test strip cassette facing upward;
hand-placing a reader onto the test strip cassette with a sensing aperture of the reader aligned with the test line of the test strip;
obtaining an assay result from the reader.

31. The method of claim 30 comprising aligning the sensing aperture of the reader with the test line of the test strip by engaging a registration feature on the bottom of the reader with a registration feature on the top of the test strip cassette.

32. The method of claim 30, wherein the reader comprises a housing, and wherein the test strip cassette is not placed inside the housing.

33. (canceled)

34. (canceled)

35. (canceled)

36. A reader for a lateral flow assay, the reader comprising:
a light source configured to illuminate an assay test strip;
a first optical sensor configured to receive light emitted from the light source that is reflected from the assay test strip;
a second optical sensor configured to receive light emitted by the light source that is not reflected from the assay test strip.

37. The reader of claim 36, comprising a controller configured to control the light source based at least in part on an output signal from the second optical sensor.

38. The reader claim 36, wherein the reader comprises a housing with at least one sensing aperture in a lower portion of the housing configured to allow light from the light source to exit the lower portion of the housing and to allow light reflected from the assay test strip to enter the lower portion of the housing and illuminate the first optical sensor.

39. The reader of claim 36, wherein the light source comprises a plurality of light sources.
